(19) United States
(12) Patent Application Publication
(10) Pub. No.: US 2022/0257922 A1
(43) Pub. Date: Aug. 18, 2022

(54) A CARDIAC PUMP

(71) Applicant: Calon Cardio-Technology Ltd., Swansea (GB)

(72) Inventors: Matthew Paul Biginton, Swansea (GB); Sajal Gurung, Swansea (GB); Robyn Williams, Swansea (GB)

(73) Assignee: Calon Cardio-Technology Ltd., Swansea (GB)

(21) Appl. No.: 17/608,007

(22) PCT Filed: Apr. 9, 2020
(86) PCT No.: PCT/EP2020/060218

§ 371 Date: Nov. 1, 2021

(30) Foreign Application Priority Data
May 2, 2019 (GB) 1906159.7

(51) Int. Cl. A61M 60/825; A61M 60/422; A61M 60/81; A61M 60/178; A61M 60/226; A61M 60/237; A61M 60/82

(52) U.S. Cl. CPC A61M 60/825; A61M 60/422; A61M 60/81; A61M 60/82; A61M 60/226; A61M 60/237; A61M 60/178

(57) ABSTRACT

A cardiac pump (1) comprising a cardiac pump housing (7), a cardiac pump rotor (8), and a bearing assembly (23), the bearing assembly (23) being configured to support the cardiac pump rotor (8) within the cardiac pump housing (7) for rotation about a rotational axis (A-A) of the rotor (8), wherein the cardiac pump rotor (8) comprises a tip profile having rotational variance about the rotational axis (A-A).

### TECHNICAL FIELD

The present disclosure relates to a cardiac pump having an asymmetric geometry and is particularly, although not exclusively, concerned with a cardiac pump having an asymmetric rotor tip.

### BACKGROUND

Advanced heart failure is a major global health problem resulting in many thousands of deaths each year and those with the disease endure a very poor quality of life. The treatment options for advanced heart failure, for example drug therapy and cardiac resynchronization (pacemakers), have generally proved unsuccessful and the only option remaining for the patients is heart transplantation. Unfortunately, the number of donor hearts available only meets a fraction of the demand, leaving many people untreated.

Ventricular Assist Devices (VAD) have been gaining increased acceptance over the last decade as an alternative therapy to heart transplantation. The use of VADs has shown that, in most cases, once the device has been implanted, the disease progression is halted, the symptoms of heart failure are relieved, and the patient regains a good quality of life.

VADs can be considered as a viable alternative to treat heart failure and offer hope to the many thousands of heart failure patients for whom a donor heart will not be available.

In general terms, it is known to provide a cardiac pump, such as a VAD, that is suitable for implantation into a ventricle of a human heart. The most common type of these implantable pumps is a miniaturised rotary pump, owing to their small size and mechanical simplicity/reliability. Such known devices have two primary components: a cardiac pump housing, which defines a cardiac pump inlet and a cardiac pump outlet; and a cardiac pump rotor, which is housed within the cardiac pump housing, and which is configured to impart energy to the fluid.

A requirement for the cardiac pump, therefore, is a bearing system that rotatably supports the cardiac pump rotor within the cardiac pump housing. Bearings systems for cardiac pumps, and generally all rotating machines such as pumps and motors, ideally achieve the fundamental function of permitting rotation of the rotor, whilst providing sufficient constraint to the rotor in all other degrees of freedom, i.e. the bearing system must support the rotor axially, radially and in pitch/yaw.

Desirable functions of bearing systems generally may include low rates of wear and low noise and vibration, and in the case of blood pumps, elimination of features that trap blood, or introduce shear stress or heat in the blood.

In known devices, the cardiac pump rotor may be rotatably supported within the housing using one of a number of different types of bearing systems. In general, there are three types of bearing systems that are utilised in cardiac pumps.

Some cardiac pumps use blood-immersed contact bearings, for example a pair of plain bearings, to rigidly support the rotor within the housing. However, for such plain bearing systems it may be difficult to ensure that the rotor is perfectly entrapped within the contact bearings. Moreover, blood-immersed contact bearings of the prior art may be susceptible to proteinaceous and other biological deposition in the bearings, and also in the region proximate to the bearings and on supporting structures around the bearings.

Other cardiac pumps use non-contact hydrodynamic bearing systems, in which the rotor is supported on a thin film of blood. In order to produce the required levels of hydrodynamic lift, hydrodynamic bearing systems require small running clearances. As a consequence, blood that passes through those small running clearances may be subjected to high levels of shear stress, which may have a detrimental effect on the cellular components of the blood, for example by causing haemolysis or platelet activation which may further lead to thrombosis.

Cardiac pumps may also employ non-contact magnetic bearing systems, in which the running clearances between the rotor and the housing may be designed such that large gaps can exist in the bearing and therefore shear-related blood damage in the bearing is reduced. However, it is common to use a passive magnetic bearing system in combination with another manner of support in at least one degree-of-freedom, for example active magnetic control, which may significantly increase the size and complexity of the design, and/or hydrodynamic suspension, which may increase the requirements with regard to manufacturing tolerances.

A common problem in all cardiac pumps is flow stasis, and cardiac pumps are carefully designed to manage all areas of flow within the pump. One area in particular where flow stasis may occur is in the region of flow that surrounds a bearing of the cardiac device. It is desirable, therefore, to disrupt any areas of flow stasis that may occur in the region of flow that surrounds a bearing during operation of the cardiac pump.

### STATEMENTS OF INVENTION

According to an aspect of the present disclosure, there is provided a cardiac pump comprising a cardiac pump housing, a cardiac pump rotor and a bearing assembly, the bearing assembly being configured to support the cardiac pump rotor within the cardiac pump housing for rotation about a rotational axis of the rotor, wherein the cardiac pump rotor comprises a tip profile having rotational variance about the rotational axis.

The rotor may comprise a rotor tip profile having rotational asymmetry about the rotational axis. The tip profile may be disposed at an end of the rotor proximal to an inlet of the cardiac pump. The rotor tip profile may be configured to direct blood across a bearing assembly.

The rotor tip profile may comprise a protrusion. The protrusion may be configured asymmetrically about the rotational axis of the rotor. The protrusion may be configured to direct blood radially and axially across a bearing assembly.

The rotor may comprise a cutaway portion. The protrusion and cutaway portion may be disposed on substantially opposite sides of the rotor tip.

The bearing assembly may comprise a contact bearing.

The cardiac pump may comprise an inlet symmetric about the rotational axis of the pump. The cardiac pump may comprise an inlet offset from the rotational axis of the pump.

The rotor may be subjected to an axial pre-load force. The axial pre-load force may be caused by a magnetic offset.

The rotor tip profile may be configured to direct blood across a feature of the pump other than a bearing assembly.

According to another aspect of the present disclosure there is provided a cardiac pump comprising a cardiac pump housing, a cardiac pump rotor and a bearing assembly, the bearing assembly being configured to support the cardiac pump rotor within the cardiac pump housing for rotation about a rotational axis of the rotor, wherein the cardiac pump rotor comprises a tip profile having rotational asymmetry about the rotational axis.

According to another aspect of the present disclosure there is provided a cardiac pump comprising a cardiac pump housing, a cardiac pump rotor and a bearing assembly, the bearing assembly being configured to rotatably support the cardiac pump rotor within the cardiac pump housing, wherein the cardiac pump rotor comprises a tip profile which is asymmetrical about at least one longitudinal plane of the rotor.

To avoid unnecessary duplication of effort and repetition of text in the specification, certain features are described in relation to only one or several aspects or embodiments of the invention. However, it is to be understood that, where it is technically possible, features described in relation to any aspect or embodiment of the invention may also be used with any other aspect or embodiment of the invention.

### BRIEF DESCRIPTION OF THE DRAWINGS

For a better understanding of the present invention, and to show more clearly how it may be carried into effect, reference will now be made, by way of example, to the accompanying drawings, in which:

FIG. 1 shows a cut-away of a heart with a cardiac pump implanted into the left ventricle;

FIG. 2 is a cross sectional view of the cardiac pump in an assembled configuration with a conventional rotor tip design;

FIG. 3 shows a partial cross sectional view of the cardiac pump of FIGS. 1 and 2 with a modified rotor tip design in which the rotor tip has rotational variance; and

FIG. 4 shows a partial cross sectional view of the cardiac pump of FIG. 3.

### DETAILED DESCRIPTION

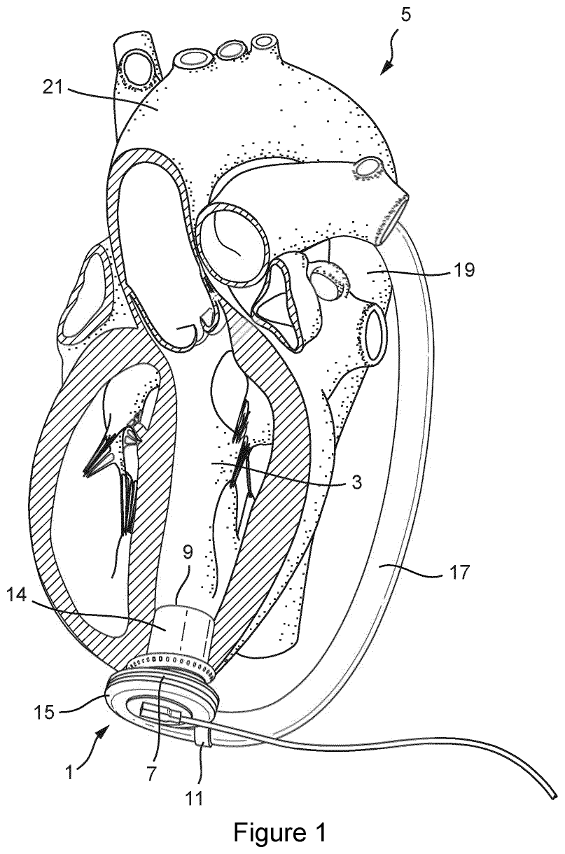

FIG. 1 depicts a cardiac pump 1 for the treatment of heart failure, for example a Ventricular Assist Device (VAD), in an implanted configuration in the left ventricle 3 of a heart 5. The cardiac pump 1 according to the present disclosure may be any appropriate type of cardiac pump. For example, the cardiac pump 1 may be an axial flow cardiac pump, a radial flow cardiac pump, or a mixed flow cardiac pump. It is understood, therefore, that where technically possible, features described in relation to a radial flow cardiac pump may be employed in any type of cardiac pump, such as an axial flow cardiac pump. Further, whilst FIG. 1 depicts the cardiac pump in an implanted configuration in the left ventricle 3 of a heart 5, it is understood that the cardiac pump 1 may be implanted in any appropriate position, for example extracorporeally, completely outside of the heart 5, or completely inside of the heart 5.

The cardiac pump 1 of FIG. 1 comprises a cardiac pump housing 7 comprising an inlet 9 for blood and an outlet 11 for blood. The cardiac pump 1 comprises a cardiac pump rotor 8 disposed at least partially within the cardiac pump housing 7. The cardiac pump rotor 8 is supported, for example rotatably supported, by way of one or more bearing assemblies 23, as described below.

The cardiac pump 1 comprises an inflow tube, for example an inflow cannula 14, which is integral to the cardiac pump housing 7. When the cardiac pump is in an implanted state, the inflow cannula 14 is situated at least partially inside the left ventricle 3, with a pumping chamber 15 being situated outside of the heart 5. The inflow cannula 14 extends between the pumping chamber 15, through the wall of the left ventricle 3 into the chamber of the left ventricle 3, so that the inlet 9 is situated completely within the left ventricle 3. The pumping chamber 15 is situated on the apex of the left ventricle 3 with the outlet 11 connected to a separate outflow cannula 17. In the example shown in FIG. 1, the outflow cannula 17 is anastomosed to a descending aorta 19, although in an alternative example the outflow cannula 17 may be anastomosed to an ascending aorta 21.

Although not shown in any of the figures, the cardiac pump 1 may comprise a magnetic drive coupling, for example a brushless DC motor. The cardiac pump rotor 8 may comprise a first portion of the magnetic drive coupling, for example one or more permanent magnets. The cardiac pump housing 7 may comprise a second portion of the magnetic drive coupling, for example one or more electrical windings. The magnetic drive coupling may be a radial magnetic drive coupling, e.g. a radial flux gap electric motor, although it is appreciated that the magnetic drive coupling may be of any appropriate configuration.

One of the most important factors in the design of a VAD is the passage of blood through the cardiac pump 1, particularly the passage of blood in the region of the bearings. The regions of blood flow around the bearings, i.e. the regions around the circumferential transition between the rotating and stationary components of a plain bearing assembly, may be areas of flow stasis and therefore predisposed to thrombus formation or indeed any type of protein deposition. It is particularly important, therefore, that bearings are well washed with a constant supply of fresh blood as the heat generated and geometrical constraints in these areas make them particularly prone to thrombus formation and/or protein deposition.

Therefore, it is desirable to directly expose one or more surfaces of the bearing, for example the interface between rotating and stationary components of the bearing, to a continuous supply of blood flow, such that the proteinaceous and cellular components of the blood responsible for pump deposition and thrombus formation are prevented from aggregating in this region.

The present disclosure relates to a cardiac pump 1 that reduces the risk of damage to the cellular components of the blood. For example, the cardiac pump 1 according to the present disclosure may mitigate the deposition of proteins and/or the formation of thrombi within the cardiac pump 1, and in particular, may mitigate the deposition of proteins and/or the formation of thrombi in areas proximate to one or more bearing assemblies of the cardiac pump 1.

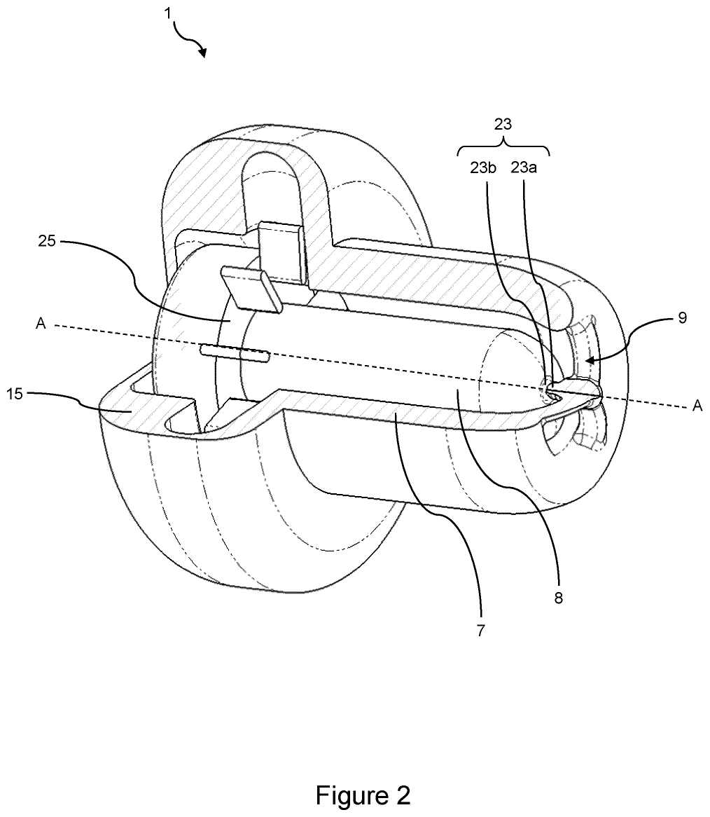

With reference to FIG. 2, the cardiac pump housing 7 is configured to rotatably support a cardiac pump rotor 8 at least partially within the cardiac pump housing 7. The cardiac pump rotor 8 is rotatably coupled to an impeller portion 25 which is configured to pump the blood and which may be provided at or towards an end of the cardiac pump rotor 8. The cardiac pump rotor 8 may be supported by one or more types of appropriate bearing assemblies, such that the cardiac pump rotor 8 is substantially constrained, e.g. in five degrees-of-freedom, and the cardiac pump rotor 8 may rotate about the rotational axis A-A. In other words, a bearing system of the cardiac pump 1 permits rotation of the cardiac pump rotor 8, which is a fundamental function of the bearing system, and provides sufficient constraint to the cardiac pump rotor 8 in all other degrees of freedom. In this manner, the bearing system supports the cardiac pump rotor 8 in the axial and radial directions, as well as in pitch and yaw. For example, the cardiac pump rotor 8 may be supported by a first plain bearing assembly and a second plain bearing assembly. Additionally or alternatively, the cardiac pump 1 may comprise one or more magnetic bearing assemblies and/or one or more electromagnetic bearing assemblies.

In the example shown in FIGS. 2 to 4, the cardiac pump rotor 8 is partially supported by a plain bearing assembly 23 located towards the inlet 9 of the cardiac pump, and it is understood that the plain bearing assembly 23 supports the cardiac pump rotor 8 in combination with at least one other bearing assembly. It is appreciated, however, that any of the bearing assemblies of the cardiac pump 1 may be positioned at any appropriate portion of the cardiac pump 1, dependent upon the operational requirements of the cardiac pump 1.

The plain bearing assembly 23 is a type of contact bearing assembly in which the bearing surfaces of the plain bearing assembly 23 are configured to be in contact during operation of the cardiac pump 1. For example, the plain bearing assembly 23 may comprise no intermediate rolling elements, i.e. motion is transmitted directly between two or more contacted surfaces of respective portions of the plain bearing assembly 23.

The plain bearing assembly 23 comprises a first plain bearing portion 23a. The first plain bearing portion 23a is coupled to the cardiac pump rotor 8 such that, during operation of the cardiac pump 1, the first plain bearing portion 23a does not rotate with the cardiac pump rotor 8. In FIGS. 2 to 4, the first plain bearing portion 23a is integral to the cardiac pump housing 7, although in an alternative example (not shown) the first plain bearing portion 23a may be a separate component rigidly fixed to the cardiac pump housing 7. In another example, the first plain bearing portion 23a may be movably coupled, for example threadably coupled, to the cardiac pump housing 7 such that the position of the first plain bearing portion 23a may be adjusted relative to the cardiac pump housing 7. The first plain bearing portion 23a may be constructed from a different material to the cardiac pump housing 7, e.g. a ceramic material. Alternatively, the first plain bearing portion 23a may be constructed from a similar material to the cardiac pump housing 7, e.g. a titanium alloy. The first plain bearing portion 23a may comprise a surface coating and/or may have had a surface treatment to improve the wear characteristics of the plain bearing assembly 23.

The plain bearing assembly 23 comprises a second plain bearing portion 23b. The second plain bearing portion 23b is coupled to the cardiac pump rotor 8 such that, during operation of the cardiac pump 1, the second plain bearing portion 23b rotates with the cardiac pump rotor 8. In the example shown in FIGS. 2 to 4, the second plain bearing portion 23b is integral to the cardiac pump rotor 8, although in an alternative example the second plain bearing portion 23b may be a separate component rigidly fixed to the cardiac pump rotor 8. In another example, the second plain bearing portion 23b may be movably coupled, for example threadably coupled, to the cardiac pump rotor 8 such that the position of the second plain bearing portion 23b may be adjusted relative to the cardiac pump rotor 8. The second plain bearing portion 23b may be constructed from a different material to the cardiac pump rotor 8, e.g. a ceramic material. Alternatively, the second plain bearing portion 23b may be constructed from a similar material to the cardiac pump rotor 8, e.g. a titanium alloy. The second plain bearing portion 23b may comprise a surface coating and/or may have had a surface treatment to improve the wear characteristics of the plain bearing assembly 23. The first and second plain bearing portions 23a, 23b may be constructed from different materials to each other, for example the first and second plain bearing portions 23a, 23b may each be constructed from a different ceramic material.

The first and second plain bearing portions 23a, 23b are configured to engage each other so as to be in contact when the cardiac pump rotor 8 and the cardiac pump housing 7 are in an assembled configuration, such that the plain bearing assembly 23 is configured to rotatably support the cardiac pump rotor 8 within the cardiac pump housing 7. In the example shown in FIGS. 2 to 4, the first and second bearing portions 23a, 23b each comprise a substantially planar articular bearing surface arranged perpendicularly to the rotational axis A-A. In this manner, the first and second bearing portions 23a, 23b are configured to support the cardiac pump rotor 8 within the cardiac pump housing 7 in an axial direction of the cardiac pump rotor 8.

The first plain bearing portion 23a may comprise a spherical segment, i.e. a truncated spherical cap or spherical frustum. The second plain bearing portion 23b may be substantially disc-shaped. It is appreciated, however, that the first and second bearing portions 23a, 23b may be of any suitable form that permits the plain bearing assembly 23 to support the cardiac pump rotor 8 in at least the axial direction, for example, the first and/or second plain bearing portions 23a, 23b may comprise a frustoconical portion.

In an alternative example, the first and second bearing portions 23a, 23b may be arranged in any suitable manner such that the plain bearing assembly 23 is configured to support the cardiac pump rotor 8 within the cardiac pump housing 7 in at least a radial direction of the cardiac pump rotor 8. As such, the bearing surfaces of the first and second bearing portions 23a, 23b may be of any appropriate form. In one example, plain bearing assembly 23 may be configured to support the cardiac pump rotor 8 in the axial direction and in the radial direction, e.g. the first and second bearing portions 23a, 23b may comprise one or more curved, e.g. partially spherical, or conical bearing surfaces configured to be in rotatable contact. For example, the plain bearing assembly 23 may comprise an at least partial ball and socket bearing, wherein the one or more bearing surfaces of the first and second bearing portions 23a, 23b are substantially conformal. In general, the plain bearing assembly 23 may be configured such that the cardiac pump rotor 8 is substantially constrained in up to five degrees-of-freedom by any combination of point-, line- or surface-contact between the bearing surfaces of the first and second bearing portions 23a, 23b.

The area of contact between the first and second bearing portions 23a, 23b may be optimised with regard to heat generation and wear characteristics of the plain bearing assembly 23. For example, the area of contact may be a substantially circular contact area having an appropriate diameter that may be selected dependent upon operational characteristics of the cardiac pump 1 and the material from which the first and/or second bearing portions 23a, 23b are fabricated. In one example, the substantially circular contact area may have a diameter within a range of approximately 10 μm to 3 mm, or, in particular, within a range of approximately 300 μm to 1 mm. It is appreciated, however, that the shape of the contact area may be of any appropriate form and/or size. In another example, the plain bearing assembly 23 may comprise a plurality of contact areas, which may each be optimised to provide the desired levels of heat generation and wear characteristics.

Typically, cardiac pumps comprising contact bearings may struggle to provide sufficient radial flow of fresh blood across the bearing surface such that deposition and aggregation may be prevented.

In view of the above discussion, it has been appreciated by the inventors that one important factor in the design of the cardiac pump 1 is the flow regime of the blood around the plain bearing assembly 23. For example, it is desirable to position the plain bearing assembly 23 in such a manner that it is exposed to a continuous flow of fresh blood for the purposes of washing the plain bearing assembly 23, for example the bearing interface of the plain bearing assembly 23, and disrupting any areas of flow stasis that may exist.

For example, the non-rotating portion 23a of the plain bearing assembly 23 may be supported by a web-like or cage-like structure disposed within the inlet cannula 14 of the cardiac pump 1. In this manner, the bearing interface of the plain bearing assembly 23 is exposed to a high flow rate of blood, which serves to help wash the bearing interface and minimise the risk of protein deposition.

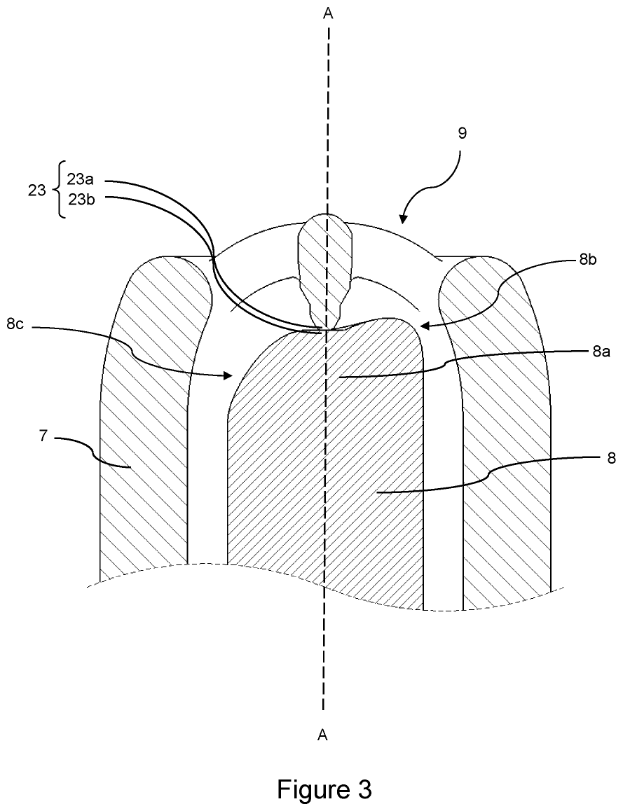

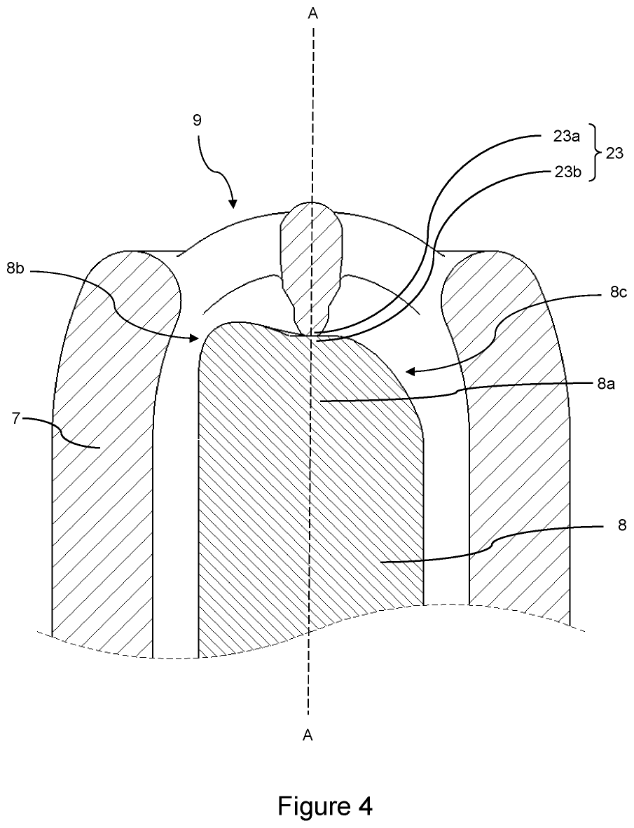

FIGS. 3 and 4 show cross sectional views through part of the cardiac pump 1, specifically the upper part of the cardiac pump 1, comprising the plain bearing assembly 23, the pump rotor 8, and part of the cardiac pump housing 7. The pump rotor 8 comprises a rotor tip 8a.

It is known for cardiac rotor tips to have a tip profile shaped similarly to a frustum or segment of a sphere (e.g. a profile representing a portion of a sphere that has been cut by two parallel planes to form the shape between the parallel planes), or a cylinder with fillet edges (e.g. the edges of the upper surface of the cylinder having been rounded to remove the sharp edge). The central and uppermost surface of each of these prior art rotor tip profiles is often perpendicular with respect to the longitudinal rotation axis, thus forming a ‘flat’ surface for the provision of a bearing assembly. These frusto-spherical and fillet-edge rotor tip profiles comprise rotational invariance, specifically rotational symmetry. In this context, rotational invariance should be taken to mean that, viewing the rotor tip 8a end on along the rotational axis A-A of the rotor 8, the profile of the rotor tip 8a is symmetric and its profile as viewed side on to the rotor 8 in a direction perpendicular to the rotational axis A-A does not vary with angle of rotation. As such, during rotation of the rotor 8, the shape of the volume that the rotor tip 8a sweeps out does not vary with position of the rotor 8.

Referring to FIG. 3, the rotor tip 8a of the present disclosure differs from those of the prior art. The rotor tip 8a of the present disclosure is provided with rotational variance. In this context, rotational variance should be taken to mean that, viewing the rotor tip 8a end on along the rotational axis A-A of the rotor 8, the profile of the rotor tip 8a is asymmetric and its profile viewed side on to the rotor in a direction perpendicular to the rotational axis A-A varies during rotation of the rotor tip 8a, i.e. the longitudinal extent of the rotor tip 8a varies around its circumference about the axis of rotation A-A. In particular, this rotational variance may be caused by an asymmetry; the rotor tip 8a may comprise a tip profile which is asymmetric about at least one longitudinal plane of the rotor 8.

As shown in FIGS. 3 and 4, the rotor tip 8a is asymmetric about the rotational axis A-A, for example rotationally asymmetric. The rotor tip 8a comprises a profile which has been modified from that of the prior art. The rotor tip 8a comprises a protrusion 8b which may extend longitudinally beyond the interface between the two bearing portions 23a, 23b, such that the protrusion 8b is longitudinally nearer to the inlet 9 than is the interface between the two bearing portions 23a, 23b. The central portion of the rotor tip 8a, which comprises the second bearing portion 23b, may be substantially perpendicular to the rotational axis A-A, such that the rotor is stably supported by the plain bearing 23 and as such the central portion of the rotor tip profile may be significantly similar to rotor tip profiles of the prior art.

On a side of the rotor tip 8a substantially opposite to the protrusion 8b, the rotor tip 8a may comprise a profile that again deviates from the frusto-spherical or fillet edge shapes of the prior art. Specifically, the rotor tip 8a comprises a profile that drops away from the upper surface/bearing interface more steeply and at lower radius, such that the radial clearance between the rotor tip 8a and the pump housing 7 is greater than the radial clearance on the opposite side of the rotor tip 8a, between the protrusion 8b and the cardiac pump housing 7, and indeed greater in clearance than if the rotor tip 8a were to comprise the profile of the prior art. This part of the rotor tip profile is termed the cutaway portion 8c.

The rotor tip profile of the present disclosure may be described as having lowered the tip height on one side of the bearing assembly 23 and raised the tip height on the other side of the bearing assembly 23 when compared with tip profiles of the prior art. In this way, the rotor tip 8a of the present disclosure comprises a profile which is rotationally variant, for example, rotationally asymmetric.

This particular rotor tip profile may be described, starting from the left side of the cross sectional view shown in FIG. 3, as extending monotonically in its longitudinal dimension in the direction of the pump inlet 9, until a point is reached on the right hand side, when the profile drops away. It will be understood by the skilled person the point at which the profile must drop away, in order to allow a sufficient radius of curvature such that blood components are not subjected to excessive shear forces due to sharp tip angles.

FIG. 3 shows the rotor 8 and rotor tip 8a in a first rotational orientation, whilst FIG. 4 shows the rotor 8 and rotor tip 8a in a second rotational orientation. The first and second orientations are separated by a half rotation, or 180 degrees.

In use, as the rotor 8 rotates about the rotational axis A-A, the protrusion 8b and cutaway portion 8c sweep out volumes of different shapes and sizes. For example, the protrusion 8b defines a rotational volume, within which some points will only be swept out by the rotor tip 8a once per rotation. Conversely, the cutaway portion 8c defines another rotational volume, within which some points will be swept out continually during every rotation, except when the cutaway portion 8c passes those exact points. This asymmetry produces a pressure difference over the rotor tip 8a, which encourages flow in the radial direction over the rotor tip 8a. In particular, blood is encouraged to flow radially across the bearing interface, away from the protrusion 8b and down the cutaway portion 8c of the rotor tip profile.

As such, when the rotor is in the first position shown in FIG. 3, blood is encouraged to flow from right to left across the bearing interface. A half rotation later, when the rotor is in the second position shown in FIG. 4, blood is encouraged to flow from left to right across the bearing interface. At fractions of a full rotation between the first and second position, the blood will be changing flow direction across the bearing interface in a continuous flow pattern/manner according to the positions of the protrusion 8b and cutaway portion 8c.

This changing flow sweeping cyclically across the bearing interface increases the washout of the bearing interface; in particular the bearing interface is washed out from every direction in each rotation, greatly decreasing the rate of deposition/heat accumulation at the bearing interface. Accordingly, any dead-spots in blood flow are swept out at least once per rotation.

The rotor tip 8a may comprise additional protrusions 8b, cutaway portions 8c or deviations from a typical rotor tip profile. The profile described above, comprising a protrusion 8b and a cutaway portion 8c on opposite sides of the rotational axis, may be repeated at a certain rotational/angular frequency, for example, every 90 degrees or quarter rotation, such that there may be greater than one protrusion 8b and/or greater than one cutaway portion 8c in the rotor tip 8a.

Whilst the features of asymmetry and/or rotational variance have been described in the context of FIGS. 3 and 4, other tip profiles will be possible which also exhibit rotational asymmetry and/or rotational invariance and also facilitate greater washing of the bearing interface. For example, an alternative rotor tip profile may comprise at least two protrusions 8b; none, all or any number of these protrusions 8b may extend further longitudinally than the bearing interface, whilst the remainder may not. Similarly, an alternative rotor tip 8a may comprise no protrusions 8b but at least one cutaway portion 8c. It will be understood by the skilled person how the above features can be varied and implemented such that the same benefits of washing out the bearing interface are achieved.

Whilst the asymmetric profile of the rotor tip 8a of the present disclosure has been described in the context of a symmetric inlet (e.g. an inlet that is located coaxially with the rotational axis A-A), asymmetric inlets can be used to further enhance the flow across the bearing interface. Asymmetric inlets are inlets which are disposed, for example, offset from a rotational axis of a rotor. In the present example, an asymmetric inlet would be located offset from the rotational axis A-A.

When compared with cardiac pumps having asymmetric inlets and symmetric rotor tip profiles, the cardiac pump 1 of the present disclosure, having a symmetric inlet 9 and an asymmetric profile of the rotor tip 8a, confers greater stability on the rotor 8. An offset inlet produces a constant pressure bias, and thus net force, in a constant direction on the rotor as blood is drawn into and passes through the pump. In contrast, the pressure difference acting radially across the rotor tip 8a of the present disclosure varies continuously in its direction as the rotor 8 rotates, thus producing a continuously varying net force on the rotor 8. This acts to make the rotor 8 more stable and thus more resistant to shock during movement of the user (e.g. exercise or other forms of physical exertion) when compared with the offset inlet of the prior art.

Similarly, the inlet 9 of the present disclosure may be made larger in dimensions than an asymmetric inlet, as the directionality of the blood flow through the inlet is of lower importance. This reduces the risk of suck down, in which the walls of the heart are sucked towards the pump inlet, and may allow a greater flow rate through the cardiac pump 1.

Additionally, the asymmetric rotor tip profile of the present disclosure may be combined with an asymmetric inlet. The combination of an asymmetric inlet and the asymmetric rotor tip 8a of the present disclosure may further enhance washing of the bearing interface, to further minimise the levels of thrombus formation, pump deposition and/or heat accumulation.

The combination of an asymmetric tip profile with an asymmetric inlet may act to counteract the constant net force exerted on the rotor when an asymmetric inlet is used with a symmetric rotor tip. This may make the rotor of a pump having an asymmetric inlet more stable.

The present disclosure is advantageous, therefore, as it provides a cardiac pump housing having an asymmetric rotor tip profile specially configured to improve the washing of a bearing assembly of a cardiac pump 1 by directing blood radially across the plain bearing assembly 23. In this way, the plain bearing assembly 23 is substantially washed with fresh blood in both an axial and a radial direction. In particular, the size, shape and/or position of the one or more protrusions 8b and/or cutaway portions 8c may be selected to provide a desired flow regime around a bearing assembly, such as a plain bearing assembly 23, of a cardiac pump 1. The rotor tip 8a may alternatively be configured to direct, for example redirect, blood towards or away from any appropriate feature of the cardiac pump 1.

The washing of the bearing assembly may be tuned depending on the desired use of the cardiac pump 1. For example, where the cardiac pump 1 is implanted into a first individual, it may be desirable to provide a first flow regime in the region surrounding the bearing assembly, and where the cardiac pump 1 is implanted into a second individual, it may be desirable to provide a second flow regime in the region surrounding the bearing assembly. The flow regime around the bearing assembly can therefore be selected depending on the condition of the individual and the physical characteristics of the individual's heart.

It will be appreciated by those skilled in the art that although the invention has been described by way of example, with reference to one or more exemplary examples, it is not limited to the disclosed examples and that alternative examples could be constructed without departing from the scope of the invention as defined by the appended claims.

1-15. (canceled)

16. A cardiac pump comprising:
a cardiac pump housing;
a cardiac pump rotor; and
a bearing assembly,
the bearing assembly being configured to support the cardiac pump rotor within the cardiac pump housing for rotation about a rotational axis of the rotor, wherein the cardiac pump rotor comprises a tip profile having rotational variance about the rotational axis.

17. The cardiac pump of claim 16, wherein the rotor comprises a rotor tip profile having rotational asymmetry about the rotational axis.

18. The cardiac pump of claim 16, wherein the tip profile is disposed at an end of the rotor proximal to an inlet of the cardiac pump.

19. The cardiac pump of claim 16, wherein the rotor tip profile is configured to direct blood across the bearing assembly.

20. The cardiac pump of claim 16, wherein the rotor tip profile comprises a protrusion.

21. The cardiac pump of claim 20, wherein the protrusion is configured asymmetrically about the rotational axis of the rotor.

22. The cardiac pump of claim 20, wherein the protrusion is configured to direct blood radially and axially across a bearing assembly.

23. The cardiac pump of claim 16, wherein the rotor comprises a cutaway portion.

24. The cardiac pump rotor of claim 23, wherein the rotor tip profile comprises a protrusion and the protrusion and cutaway portion are disposed on substantially opposite sides of the rotor.

25. The cardiac pump of claim 16, wherein the bearing assembly comprises a contact bearing.

26. The cardiac pump of claim 16, wherein the cardiac pump comprises an inlet symmetric about the rotational axis of the pump.

27. The cardiac pump of claim 16, wherein the cardiac pump comprises an inlet offset from the rotational axis of the pump.

28. The cardiac pump of claim 16, wherein the rotor is subjected to an axial pre-load force.

29. The cardiac pump of claim 28, wherein the axial pre-load force is caused by a magnetic offset.

30. The cardiac pump of claim 16, wherein the rotor tip profile is configured to direct blood across a feature of the pump other than a bearing assembly.
